(19) United States
(12) Patent Application Publication
(10) Pub. No.: US 2017/0319875 A1
(43) Pub. Date: Nov. 9, 2017

(54) SYSTEM AND METHOD FOR PRETREATMENT OF A VOLUME OF TISSUE SLATED FOR TREATMENT

(71) Applicant: SonaCare Medical, LLC, Charlotte, NC (US)

(72) Inventors: Mark Carol, Cornelius, NC (US); Hashim U. Ahmed, London (GB)

(21) Appl. No.: 15/585,383

(22) Filed: May 3, 2017

Related U.S. Application Data
(60) Provisional application No. 62/331,161, filed on May 3, 2016.

(51) Int. Cl. A61N 7/02; A61B 5/00; G06T 7/00; A61B 90/00; A61B 8/08; A61B 18/00

(52) U.S. Cl. CPC A61N 7/02; A61B 8/52; A61B 5/4848; A61B 8/08; G06T 7/0016; A61B 90/37; A61B 2018/00577; A61B 2090/378; G06T 2207/10132

(57) ABSTRACT

A method for delivering therapeutic ultrasound to a patient to ensure full treatment of targeted tissue can include performing preoperative imaging of a first volume of targeted tissue of a patient using an ultrasound probe and creating a first treatment plan. Energy can be delivered into at least a distal portion of the first volume. The amount of energy delivered can be sufficient to produce swelling of tissue in the first volume. The first volume can be reimaged to identify if any changes have occurred in at least one of a size, shape and location of the first volume of the targeted tissue. A second treatment plan can be designed to treat a second volume of tissue equivalent to the changed first volume of targeted tissue. Energy can be delivered into the second volume of the targeted tissue.

### CROSS-REFERENCE TO RELATED APPLICATIONS

The present application claims priority to U.S. Provisional Application No. 62/331,161, filed May 3, 2016 and titled “System and Method for Pretreatment of a Volume Slated for Treatment,” which is herein incorporated by reference in its entirety.

### SUMMARY

Focused ultrasound (“US”) ablation delivers mechanical and thermal energy to tissue that can lead to irreversible coagulative necrosis secondary to the high temperatures generated in the target tissue. Nonablative focused US delivers mechanical and thermal energy to tissue that that causes reversible changes in cell function or structure. Focused US is delivered typically using a series of “shots” of dose placed in a precise pattern that combine to treat the entire targeted volume of tissue. These shots can be grouped into zones of treatment. The zone furthest from the treating transducer is delivered first, with zones progressively closer to the transducer being treated in turn. This order of treatment is preferred because the process of dose delivery, and especially tissue ablation, changes the characteristics of the tissue such that is difficult or impossible to treat through ablated tissue in the immediate period following energy deposition in intervening tissue.

It is known, and has been has been reported in the literature, that the delivery of doses of focused US can cause swelling and anatomic distortion of targeted tissue. Van Leenders et al evaluated histopathological changes in nine patients treated with radical prostatectomy immediately after high intensity focused ultrasound (HIFU). See “Histopathological changes associated with intensity focused ultrasound (HIFU) treatment for localized adenocarcinoma of the prostate,” which is incorporated herein by reference in its entirety. Van Leenders found incomplete cell destruction, especially in the ventral, lateral and dorsal aspects of the treated prostate lobe. As analyzed by conventional, immunohistochemical, and ultrastructural microscopy, Van Leenders concluded that HIFU does not affect the whole area treated, leaving untreated vital tissue at the ventral and lateral sides of the prostate. Shoji et al concluded that the incomplete treatments most likely are due to skip lesions produced as a result of relative tissue movement during the course of a treatment based on their clinical findings of swelling and shifts in the ventral and lateral direction in patients treated with focused ultrasound. See “Prostate Swelling and Shift During High Intensity Focused Ultrasound: Implication for Targeted Focal Therapy,” which is incorporated herein by reference in its entirety. Shoji further documented that most prostate swelling and relative positional changes occur during the initial portion of a treatment session; once sufficient heat has been delivered to the gland, further changes in size, shape, and location are minimal. Shoji identified further that monitoring treatment such that areas that have been underdosed for any reasons are retreated, which improves outcomes in terms of disease control.

As noted, changes in size, shape, and/or location can result in a poor outcome. However, it is very difficult to track specific tissue while or once it has been treated, or to determine whether portions of tissue have been treated adequately. Implanted fiducials, the standard or prior art means for tracking tissue movement during a noninvasive intervention such as radiation therapy, can interfere with dose delivery. Imaging software for the most part is not sophisticated enough to differentiate directly between treated and untreated tissue at the histological level. Algorithms exist, such as the Tissue Change Monitoring (TCM) algorithm described in U.S. Pat. No. 8,235,902, which is incorporated by reference herein in its entirety, that can be used to assess the adequacy of dose delivery to a targeted volume of tissue such that retreatment of that region can be performed if required in order to deliver the desired dose and were used by Shoji. However, such algorithms cannot differentiate specifically whether the imaged tissue that was treated or undertreated is the tissue that was targeted originally, as the original targeted tissue can move in and out of the imaging field.

Therefore, what is desirable and provided by the presently disclosed technology is a new approach to the ablation of targeted tissue using therapeutic US that ensures that all or substantially all tissue is treated fully.

In one embodiment of the present disclosure, a method for delivering a surgical treatment is disclosed that includes performing preoperative imaging and treatment planning to identify a target volume to treat, delivering enough energy into a most distal portion of the volume sufficient to produce tissue swelling, reimaging the volume in order to identify a new target volume to treat, creating a new plan designed to treat the new target volume, and/or delivering a treatment to the entire new target volume. The steps of preoperative imaging and treatment can be completed with a single probe, or with a treatment probe and a separate imaging device. The disclosed method can include the step of ablating a most distal portion of the volume. The disclosed method can include performing an image fusion of more than one image set from the same or different imaging modalities in order to relate the actual targeted volume to a predetermined desired target volume. The disclosed method can also include interrogating the tissue to determine whether there are regions that have been ablated completely such that the regions may not need to be retreated. The term “interrogating” is broadly defined herein to include at least (i) imaging, (ii) using one or more software algorithms to analyze ultrasound imaging data, (iii) using other tools such as RF, NIR or US to analyze tissue, (iv) measure tissue impedance, (v) measure tissue elasticity, (vi) measure tissue temperature, and/or (vii) any means of tissue analysis developed after the filing of present application. The disclosed method can further include the steps of reimaging, replanning, and retreating at additional stages during the course of a complete treatment so as to compensate for additional changes that may occur during the course of a complete treatment.

In another embodiment, the presently disclosed technology is directed to a method for delivering therapeutic ultrasound to a patient to ensure full treatment of targeted tissue. The method can include the steps of: a) performing preoperative imaging of a first volume of targeted tissue of a patient using an ultrasound probe; b) creating a first treatment plan based on the results of step a); c) delivering energy, via the ultrasound probe, in accordance with the first treatment plan into at least a distal portion of the first volume, the amount of energy delivered by the ultrasound probe being sufficient to produce swelling of tissue in the first volume; d) reimaging the first volume using the ultrasound probe to identify if any changes have occurred in at least one of a size, shape and location of the first volume of the targeted tissue; e) creating a second treatment plan designed to treat a second volume of tissue equivalent to the changed first volume of targeted tissue based on the results of step d); and f) delivering energy, via the ultrasound probe, in accordance with the second treatment plan into the second volume of the targeted tissue.

In yet another embodiment, the presently disclosed technology is directed to a method delivering ultrasound to a patient to ensure full treatment of targeted tissue. The method can include the steps of: a) performing preoperative imaging of a first volume of targeted tissue of a patient using an ultrasound probe; b) creating a first treatment plan based on the results of step a), the first treatment plan being determined by calculating an amount of heat required to be delivered to the first volume to cause the first volume to swell; c) delivering energy, via the ultrasound probe, in accordance with the first treatment plan into at least a portion of the first volume that is distal to the probe, the amount of energy delivered by the ultrasound probe being sufficient to produce swelling of tissue in the first volume; d) reimaging the first volume using the ultrasound probe to identify if any changes have occurred in the first volume of the targeted tissue, any changes being identified by comparison of two or more image data sets from the ultrasound probe; e) creating a second treatment plan designed to treat a second volume of the targeted tissue based on the results of step d); and f) delivering energy, via the ultrasound probe, in accordance with the second treatment plan into the second volume of the targeted tissue. The second volume of the targeted tissue at least partially overlaps with the first volume of the targeted tissue.

In a further embodiment, the presently disclosed technology is directed to a system for delivering ultrasound to a patient to ensure full treatment of targeted tissue. The system can include one or more processors and one or more memories operatively coupled to the one or more processors and having computer readable instructions stored thereon which, when executed by at least one of the one or more processors, causes the at least one of the one or more processors to: a) perform preoperative imaging of a first volume of targeted tissue of a patient using an ultrasound probe; b) create a first treatment plan based on the results of step a); c) deliver energy, via the ultrasound probe, in accordance with the first treatment plan into at least a portion of the first volume that is distal to the probe, the amount of energy delivered by the ultrasound probe being sufficient to produce swelling of tissue in the first volume; d) reimage the first volume using the ultrasound probe to identify if any changes have occurred in the first volume of the targeted tissue; e) create a second treatment plan designed to treat a second volume of the targeted tissue based on the results of step d); and f) deliver energy, via the ultrasound probe, in accordance with the second treatment plan into the second volume of the targeted tissue.

In still a further embodiment, the presently disclosed technology is directed to non-transitory computer-readable medium having computer-readable code stored thereon that, when executed by one or more computing devices, can cause the one or more computing devices to: a) perform, by at least one of the one or more computing devices, preoperative imaging of a first volume of targeted tissue of a patient using an ultrasound probe; b) create, by at least one of the one or more computing devices, a first treatment plan based on the results of step a); c) deliver, by at least one of the one or more computing devices, energy, via the ultrasound probe, in accordance with the first treatment plan into at least a portion of the first volume that is distal to the probe, the amount of energy delivered by the ultrasound probe being sufficient to produce swelling of tissue in the first volume; d) reimage, by at least one of the one or more computing devices, the first volume using the ultrasound probe to identify if any changes have occurred in the first volume of the targeted tissue; e) create, by at least one of the one or more computing devices, a second treatment plan designed to treat a second volume of the targeted tissue based on the results of step d); and f) deliver, by at least one of the one or more computing devices, energy, via the ultrasound probe, in accordance with the second treatment plan into the second volume of the targeted tissue.

As is to be appreciated by one skilled in the art, one or more aspects of the foregoing disclosed systems and methods may be combined or even omitted, if desirable.

### DRAWINGS

The foregoing summary, as well as the following detailed description of the invention, will be better understood when read in conjunction with the appended drawing(s). For the purpose of illustrating the invention, there are shown in the drawings various illustrative embodiments. It should be understood, however, that the invention is not limited to the precise arrangements and instrumentalities shown.

FIG. 1 is a generalized flow chart outlining steps of one embodiment of the present disclosure;

FIG. 2 is a flow chart outlining steps of one embodiment of the present disclosure;

FIG. 3 is a graph showing a rate of change of a target volume of a patient versus time of one embodiment of the present disclosure;

FIG. 4 is a schematic diagram of a computing system of one embodiment of the present disclosure; and

FIG. 5 is a perspective view of an ultrasound probe according to one embodiment of the present disclosure.

### DETAILED DESCRIPTION

Various embodiments of the present disclosure are described hereinafter with reference to the figures. It should be noted that the figures are not drawn to scale and elements of similar structures or functions are represented by like reference numerals throughout the figures. It should also be noted that the figures are not intended to facilitate the description of specific embodiments of the invention. The figures are not intended as an exhaustive description of the invention or as a limitation on the scope of the invention. In addition, an aspect described in conjunction with a particular embodiment of the present invention is not necessarily limited to that embodiment and can be practiced in any other embodiments of the present invention. It will be appreciated that while various embodiments of the invention are described in connection with radiation treatment of tumors, the claimed invention has application in other industries and to targets other than cancers. Unless specifically set forth herein, the terms “a,” “an” and “the” are not limited to one element, but instead should be read as “at least one.”

In one embodiment, the present disclosure includes a method for performing a surgical procedure. The method can include a planned pretreatment of at least one portion of targeted tissue in order to prime the tissue by producing changes in the tissue such that further positional or geometric changes in the tissue are minimized during subsequent portions of the treatment. Energy delivered during a “priming” treatment may be ablative or nonablative as long as sufficient heat is deposited in the tissue to maximize the amount of tissue distortion such that subsequent heat delivery produces minimal additional distortion/movement.

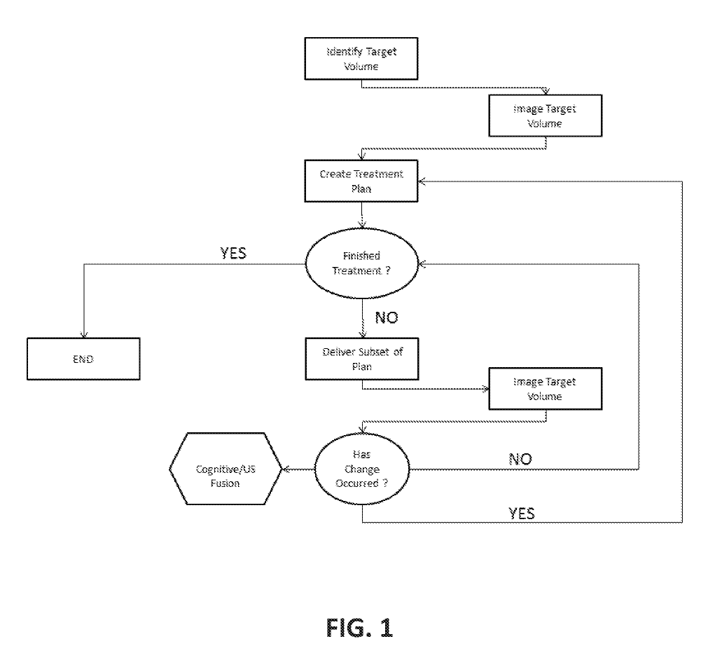

Referring to FIG. 1, in one embodiment, the present disclosure includes a method that involves at least the following steps:
1. Identifying a volume of tissue to be targeted for focused ultrasound treatment;
2. Imaging the volume using ultrasound;
3. Creating a treatment plan for ablating the volume of tissue;
4. Delivering a subset of the treatment plan to a most distal portion of the volume of tissue, the subset of the treatment plan being sufficient to cause a most distal portion of the volume of tissue to change its anatomic position and/or configuration;
5. Reimaging the volume with ultrasound in order to identify if any changes (e.g., size, shape and/or location) have occurred in a most distal portion of the volume of tissue;
6. Identifying a new target volume to treat;
7. Creating a new treatment plan designed to treat the new target volume; and/or
8. Delivering a treatment to the new target volume.

The first step of identifying described above can be based on using available diagnostic or interrogation information to decide that one area requires treatment while another does not. Multiparametric MRI can be used, as can computed tomography (CT), ultrasound (US), optical imaging, or any form of diagnostic information that differentiates between tissues. The volume of tissue identified can also be an area that the surgeon wants to ablate for anatomical reasons.

The subset of the treatment plan can be determined by selecting any portion or portions of the entire volume of tissue to be treated. In one embodiment, the subset of the treatment plan can be determined by calculating the amount of heat required to be delivered to the volume to cause the volume to swell maximally (e.g., until it swells no more with additional heat introduced) based on characteristics of the tissue being treated. In one embodiment, the amount of energy delivered at this step could be an amount of energy that is sufficient to cause irreversible damage to the tissue. However, in an alternative embodiment, it could be an amount of energy that causes only reversible damage. In one embodiment, the subset of the treatment can be an arbitrarily defined subset of the entire treatment volume.

Assessment of change can be made using cognitive registration—human comparison of two or more image datasets—or can be made using an ultrasound fusion algorithm that compares two or more data sets and identifies differences between them. In another embodiment, implanted fiducials or markers can be used to track change in position and/or shape of the targeted volume of tissue. In this approach, one or more fiducials or markers can be implanted near or in the volume, with the position of the fiducials post initial deposition of energy compared to their position predisposition of energy. In one embodiment, the fiducials implanted in such locations do not interfere with the treatment beam or are of a size such that they do not interfere with the treatment beam.

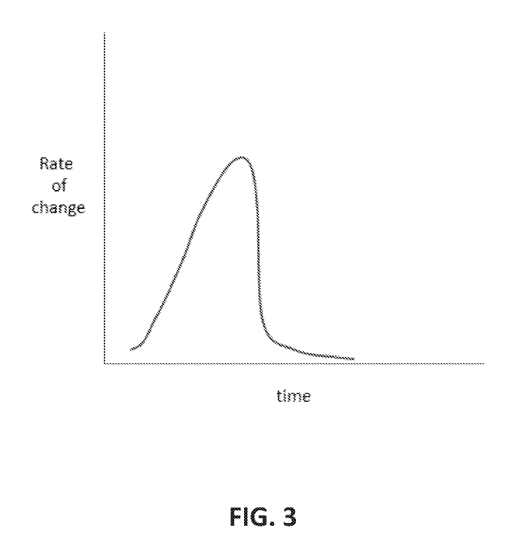

In one embodiment, the subset of the treatment plan can be determined in an iterative fashion based on using ultrasound to ultrasound (“US-US”) fusion. After an initial treatment plan is created consisting of a series of shots of dose, delivery of the plan is initiated. As dose is delivered, the volume will begin to change size, shape, and/or location. After each shot, an ultrasound dataset of the entire treatment volume can be generated and that volume can be compared to the volume that existed immediately prior to the shot. The amount of difference will start out small, will increase as more doses are delivered, and then will fall off as maximum changes in size, shape, and/or position are realized. See FIG. 3. At that point, the change in the target volume can be considered to have been fully realized, a new treatment plan based on the new treatment volume can be created, and delivery of the full plan can be undertaken. In this embodiment, intermittent or continuous assessment of any change in volume can take place.

A goal of one embodiment of the present disclosure is to treat the same, and all of the same, tissue with the new treatment plan as with the first or original treatment plan. However, due to movement, the original tissue may not be where it was originally or due to swelling the size and/or the shape of the original volume can change, thus resulting in a non-identical volume being treated with the new or second treatment plan.

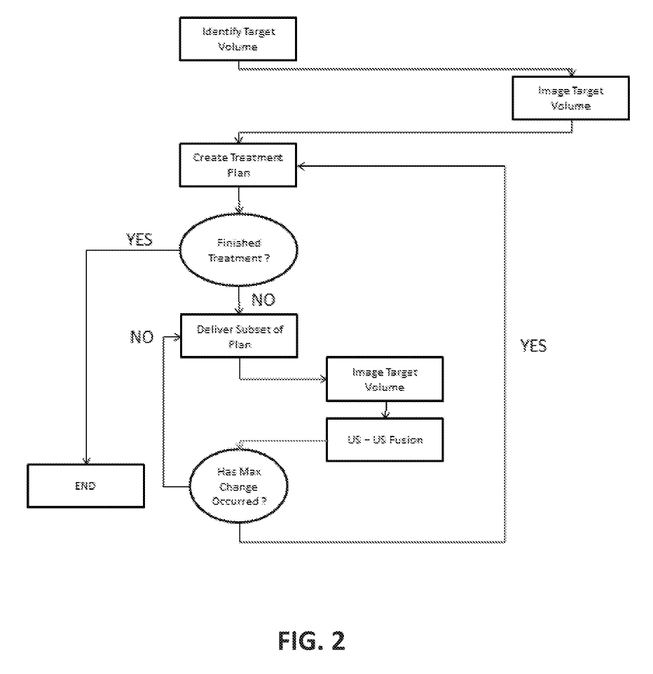

Referring to FIG. 2, in this embodiment, the steps can include:
1. Perform an initial MRI-US fusion to establish a target volume in untreated tissue, an organ or multiple organs, a subset of an organ, or the like.
2. Create a complete plan.
3. Begin to deliver the plan.
4. Automatically perform a US-US fusion after each shot or several or a couple of shots.
5. Automatically assess the amount of change.
6. The amount of change will begin small as heat is first put into the gland, will increase in magnitude as more dose of energy is delivered, and will begin to diminish as enough heat has been delivered to produce maximal change.
7. Using a predefined limit (e.g., user controllable), the systems will alert user when maximal change has occurred.
8. New treatment plan is created.
9. Complete new treatment plan is delivered.
10. Intermittent or continuous US-US fusion is performed so as to alert user if change is reoccurring and replanning should be undertaken.

When using fiducials and/or US imaging to track change, it is possible to track continuously to assess relative change as energy is delivered until change ceases to occur, at which point a new plan would be created and the definitive plan delivered. In addition, it is possible to reassess change at any point during the delivery of the complete new treatment plan. For instance, in one embodiment, if it is necessary to pause the treatment for some reason which results in a cooling of the volume, a new priming dose of energy can be delivered whose impact can be assessed prior to creating a revised plan that is delivered completely.

In an additional embodiment, a nonablative amount of energy, sufficient to cause the volume to change its anatomic position and/or configuration, but without ablating the volume, can be delivered to a distal most portion of the volume. A nonablative dose of energy can have the same impact on tissue, causing it to swell and shift position, and can be used to prime the volume prior to delivering a fully ablative dose. In a further embodiment, a nonablative amount of energy can be delivered to some portion of the volume that is distal to the location of an ultrasound probe 12 to prime the volume prior to delivering a nonablative dose of energy to the entire volume.

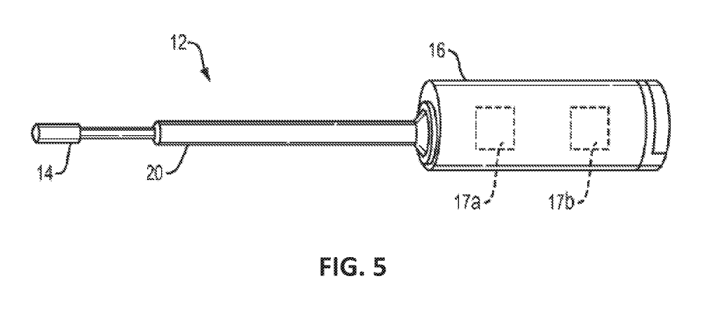

The ultrasound probe 12 can include a shaft 20 with at least one transducer 14 at a distal end thereof. The at least one transducer 14 can include at least one image (visualization) transducer and at least one therapy transducer. The image transducer can be used to generate diagnostic images of a treatment site, and the therapy transducer can deliver to a treatment site a therapeutic dose of focused ultrasound that can heat and/or disrupt tissue either permanently or transiently. A housing 16 of the probe 12 can contain electronics 17a and one or more motors 17b, which can be used to operate and/or manipulate at least portions of the probe 12 by the shaft 20. One example of an ultrasound probe that can be employed is shown in FIG. 5. Another example of an ultrasound probe that can be employed is that disclosed in U.S. Patent Application No. US 2012/0035473, which is herein incorporated by reference in its entirety.

There can be instances where the new target volume is identical to the original target volume, that is, where the delivery of energy to the volume did not result in any changes to the volume. In such a case, the original plan would continue to be delivered. In most instances, changes will occur consistent with the known physics of depositing thermal energy into tissue and will prompt the desirability to making changes in the treatment plan. The new or second volume can be larger or smaller than the original or first volume, can overlap or partially overlap the original volume, or can be displaced such that there is no overlap between the two.

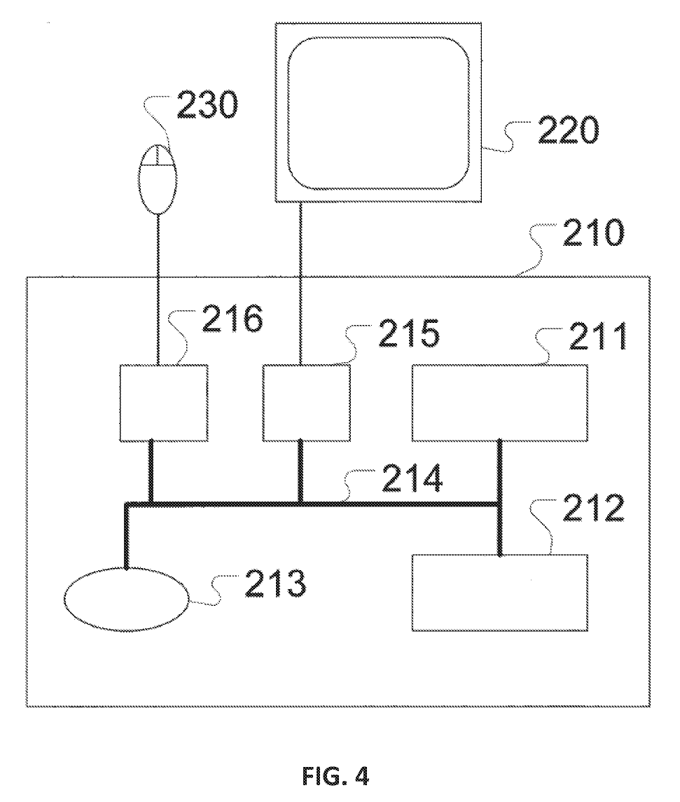

One or more of the above-described techniques and/or embodiments may be implemented with or involve software, for example modules executed on one or more computing devices 210 (see FIG. 4). Of course, modules described herein illustrate various functionalities and do not limit the structure or functionality of any embodiments. Rather, the functionality of various modules may be divided differently and performed by more or fewer modules according to various design considerations.

Each computing device 210 may include one or more processing devices 211 designed to process instructions, for example computer readable instructions (i.e., code), stored in a non-transient manner on one or more storage devices 213. By processing instructions, the processing device(s) 211 may perform one or more of the steps and/or functions disclosed herein. Each processing device may be real or virtual. In a multi-processing system, multiple processing units may execute computer-executable instructions to increase processing power. The storage device(s) 213 may be any type of non-transitory storage device (e.g., an optical storage device, a magnetic storage device, a solid state storage device, etc.). The storage device(s) 213 may be removable or non-removable, and may include magnetic disks, magneto-optical disks, magnetic tapes or cassettes, CD-ROMs, CD-RWs, DVDs, BDs, SSDs, or any other medium which can be used to store information. Alternatively, instructions may be stored in one or more remote storage devices, for example storage devices accessed over a network or the internet.

Each computing device 210 additionally may have memory 212, one or more input controllers 216, one or more output controllers 215, and/or one or more communication connections 240. The memory 212 may be volatile memory (e.g., registers, cache, RAM, etc.), non-volatile memory (e.g., ROM, EEPROM, flash memory, etc.), or some combination thereof. In at least one embodiment, the memory 212 may store software implementing described techniques.

An interconnection mechanism 214, such as a bus, controller or network, may operatively couple components of the computing device 210, including the processor(s) 211, the memory 212, the storage device(s) 213, the input controller(s) 216, the output controller(s) 215, the communication connection(s) 240, and any other devices (e.g., network controllers, sound controllers, etc.). The output controller(s) 215 may be operatively coupled (e.g., via a wired or wireless connection) to one or more output devices 220 (e.g., a monitor, a television, a mobile device screen, a touch-display, a printer, a speaker, etc.) in such a fashion that the output controller(s) 215 can transform the display on the display device 220 (e.g., in response to modules executed). The input controller(s) 216 may be operatively coupled (e.g., via a wired or wireless connection) to an input device 230 (e.g., a mouse, a keyboard, a touch-pad, a scroll-ball, a touch-display, a pen, a game controller, a voice input device, a scanning device, a digital camera, etc.) in such a fashion that input can be received from a user.

The communication connection(s) 240 may enable communication over a communication medium to another computing entity. The communication medium conveys information such as computer-executable instructions, audio or video information, or other data in a modulated data signal. A modulated data signal is a signal that has one or more of its characteristics set or changed in such a manner as to encode information in the signal. By way of example, and not limitation, communication media include wired or wireless techniques implemented with an electrical, optical, RF, infrared, acoustic, or other carrier.

FIG. 4 illustrates the computing device 210, the output device 220, and the input device 230 as separate devices for ease of identification only. However, the computing device 210, the display device(s) 220, and/or the input device(s) 230 may be separate devices (e.g., a personal computer connected by wires to a monitor and mouse), may be integrated in a single device (e.g., a mobile device with a touch-display, such as a smartphone or a tablet), or any combination of devices (e.g., a computing device operatively coupled to a touch-screen display device, a plurality of computing devices attached to a single display device and input device, etc.). The computing device 210 may be one or more servers, for example a farm of networked servers, a clustered server environment, or a cloud services running on remote computing devices.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims. It is understood that the presently disclosed technology covers any combination of the above described features and components, and any combination of the following claims.

We claim:

1. A method for delivering therapeutic ultrasound to a patient to ensure full treatment of targeted tissue, the method comprising the steps of:
a) performing preoperative imaging of a first volume of targeted tissue of a patient using an ultrasound probe;
b) creating a first treatment plan based on the results of step a);
c) delivering energy, via the ultrasound probe, in accordance with the first treatment plan into at least a distal portion of the first volume, the amount of energy delivered by the ultrasound probe being sufficient to produce swelling of tissue in the first volume;
d) reimaging the first volume using the ultrasound probe to identify if any changes have occurred in at least one of a size, shape and location of the first volume of the targeted tissue;
e) creating a second treatment plan designed to treat a second volume of tissue equivalent to the changed first volume of targeted tissue based on the results of step d); and
f) delivering energy, via the ultrasound probe, in accordance with the second treatment plan into the second volume of the targeted tissue.

2. The method of claim 1, where the second volume of the targeted tissue is not identical to the first volume of the targeted tissue.

3. The method of claim 2, wherein the second volume of the targeted tissue at least partially overlaps with the first volume of the targeted tissue.

4. The method of claim 1, wherein the first volume of the targeted tissue can be arbitrary portion the tissue of the patient.

5. The method of claim 1, wherein the first treatment plan can be determined by calculating an amount of heat required to be delivered to the first volume to cause the first volume to swell.

6. The method of claim 1, wherein step d) includes comparison of two or more image data sets from the ultrasound probe.

7. The method of claim 1, wherein step d) includes employing an ultrasound fusion algorithm that compares two or more data sets from the ultrasound probe and identifies differences between the two or more data sets.

8. The method of claim 1, further comprising:
implanting fiducials or markers near or in the first volume to track change of at least one of position and shape of the first volume.

9. The method of claim 8, wherein a position of the fiducials after step c) is compared to a position of the fiducials prior to step c).

10. The method of claim 1, wherein the energy delivered in step c) is ablative.

11. The method of claim 1, wherein the energy delivered in step c) is non-ablative.

12. The method of claim 1, wherein the energy delivered in step f) is ablative.

13. The method of claim 1, wherein the energy delivered in step f) is non-ablative.

14. The method of claim 1, wherein the changes of step c) comprise at least one of positional and geometric changes to the first volume.

15. The method of claim 1, wherein, prior to step a), a MRI-US fusion is performed of an untreated gland of the patient.

16. The method of claim 1, wherein the energy delivered in step c) as a series of discrete shots.

17. The method of claim 16, wherein ultrasound to ultrasound fusion is performed after each shot of energy delivered in step d).

18. The method of claim 1, wherein the energy delivered in step c) is continuous.

19. The method of claim 1, wherein steps c) and d) are repeated until a difference between an ultrasound dataset prior to a shot of energy and after a shot of energy is less than a preset limit.

20. A method for delivering ultrasound to a patient, the method comprising the steps of:
a) performing preoperative imaging of a first volume of targeted tissue of a patient using an ultrasound probe;
b) creating a first treatment plan based on the results of step a), the first treatment plan being determined by calculating an amount of heat required to be delivered to the first volume to cause the first volume to swell;
c) delivering energy, via the ultrasound probe, in accordance with the first treatment plan into at least a portion of the first volume that is distal to the probe, the amount of energy delivered by the ultrasound probe being sufficient to produce swelling of tissue in the first volume;
d) reimaging the first volume using the ultrasound probe to identify if any changes have occurred in the first volume of the targeted tissue, any changes being identified by employing an ultrasound fusion algorithm that compares two or more data sets from the ultrasound probe and identifies differences between the two or more data sets;
e) creating a second treatment plan designed to treat a second volume of the targeted tissue based on the results of step d); and
f) delivering energy, via the ultrasound probe, in accordance with the second treatment plan into the second volume of the targeted tissue,
wherein the second volume of the targeted tissue at least partially overlaps with the first volume of the targeted tissue, and wherein the method ensures full treatment of the targeted tissue.

21. The method of claim 20, wherein the energy delivered in step c) is ablative.

22. The method of claim 20, wherein the energy delivered in step f) is ablative.

23. The method of claim 20, wherein the energy delivered in step f) is non-ablative.

24. A method for delivering ultrasound to a patient to ensure full treatment of targeted tissue, the method comprising the steps of:
a) performing preoperative imaging of a first volume of targeted tissue of a patient using an ultrasound probe;
b) creating a first treatment plan based on the results of step a), the first treatment plan being determined by calculating an amount of heat required to be delivered to the first volume to cause the first volume to swell;
c) delivering energy, via the ultrasound probe, in accordance with the first treatment plan into at least a portion of the first volume that is distal to the probe, the amount of energy delivered by the ultrasound probe being sufficient to produce swelling of tissue in the first volume;
d) reimaging the first volume using the ultrasound probe to identify if any changes have occurred in the first volume of the targeted tissue, any changes being identified by comparison of two or more image data sets from the ultrasound probe;
e) creating a second treatment plan designed to treat a second volume of the targeted tissue based on the results of step d); and
f) delivering energy, via the ultrasound probe, in accordance with the second treatment plan into the second volume of the targeted tissue,
wherein the second volume of the targeted tissue at least partially overlaps with the first volume of the targeted tissue.

25. The method of claim 24, wherein the energy delivered in step c) is non-ablative.

26. The method of claim 24, wherein the energy delivered in step f) is ablative.

27. The method of claim 24, wherein the energy delivered in step f) is non-ablative.

28. A system for delivering ultrasound to a patient to ensure full treatment of targeted tissue, the system comprising:
one or more processors; and
one or more memories operatively coupled to the one or more processors and having computer readable instructions stored thereon which, when executed by at least one of the one or more processors, causes the at least one of the one or more processors to:
a) perform preoperative imaging of a first volume of targeted tissue of a patient using an ultrasound probe;
b) create a first treatment plan based on the results of step a);
c) deliver energy, via the ultrasound probe, in accordance with the first treatment plan into at least a portion of the first volume that is distal to the probe, the amount of energy delivered by the ultrasound probe being sufficient to produce swelling of tissue in the first volume;
d) reimage the first volume using the ultrasound probe to identify if any changes have occurred in the first volume of the targeted tissue;
e) create a second treatment plan designed to treat a second volume of the targeted tissue based on the results of step d); and
f) deliver energy, via the ultrasound probe, in accordance with the second treatment plan into the second volume of the targeted tissue.

29. A non-transitory computer-readable medium having computer-readable code stored thereon that, when executed by one or more computing devices, causes the one or more computing devices to:
a) perform, by at least one of the one or more computing devices, preoperative imaging of a first volume of targeted tissue of a patient using an ultrasound probe;
b) create, by at least one of the one or more computing devices, a first treatment plan based on the results of step a);
c) deliver, by at least one of the one or more computing devices, energy, via the ultrasound probe, in accordance with the first treatment plan into at least a portion of the first volume that is distal to the probe, the amount of energy delivered by the ultrasound probe being sufficient to produce swelling of tissue in the first volume;
d) reimage, by at least one of the one or more computing devices, the first volume using the ultrasound probe to identify if any changes have occurred in the first volume of the targeted tissue;
e) create, by at least one of the one or more computing devices, a second treatment plan designed to treat a second volume of the targeted tissue based on the results of step d); and
f) deliver, by at least one of the one or more computing devices, energy, via the ultrasound probe, in accordance with the second treatment plan into the second volume of the targeted tissue.
